(19) United States
(12) Patent Application Publication
(10) Pub. No.: US 2012/0220557 A1
(43) Pub. Date: Aug. 30, 2012

(54) LIQUID PROPELLANT-FREE FORMULATION COMPRISING AN ANTIMUSCARINIC DRUG

(75) Inventors: Annamaria Soliani RASCHINI, Parma (IT); Emilio Lutero, Parma (IT)

(73) Assignee: CHIESI FARMACEUTICI S.p.A., Parma (IT)

(21) Appl. No.: 13/370,380

(22) Filed: Feb. 10, 2012

(30) Foreign Application Priority Data
Feb. 17, 2011 (EP) 11154862.4

(51) Int. Cl. A61K 31/439; A61K 31/573; A61P 11/00; A61P 29/00; A61P 11/06

(52) U.S. Cl. 514/171; 514/305

(57) ABSTRACT

Liquid, propellant-free pharmaceutical formulation for administration by nebulization which comprising an antimuscarinic drug as active ingredient are useful for the prevention and/or treatment of a wide range of conditions including respiratory disorders.

### CROSS REFERENCES TO RELATED APPLICATIONS

This application claims priority to European Patent Application No. 11154862.4, filed on Feb. 17, 2011, which is incorporated herein by reference in its entirety.

### BACKGROUND OF THE INVENTION

1. Field of the Invention

The present invention relates to liquid propellant-free pharmaceutical formulations suitable for administration by a nebulizer, which comprise an antimuscarinic drug as an active ingredient. The present invention also relates to processes for the preparation of such a formulation and to methods for the prevention and/or treatment of a wide range of conditions including respiratory disorders by administering such a formulation.

2. Discussion of the Background

Airway obstruction characterizes a number of severe respiratory diseases including asthma and chronic obstructive pulmonary disease (COPD). Events leading to airway obstruction include edema of airway walls, increased mucous production, and inflammation.

Drugs for treating respiratory diseases such as asthma and COPD are currently administered through inhalation. One of the advantages of the inhalatory route over the systemic one is the possibility of delivering the drug directly at the site of action, avoiding any systemic side-effects, thus providing a more rapid clinical response and a higher therapeutic ratio.

An important class of therapeutic agents used as bronchodilators is represented by the muscarinic receptor antagonist inhibitors belonging to the class of the quaternary ammonium salts, and in particular by the selective M3 receptor antagonists (hereinafter M3 antagonists). For example, M3 antagonists have been disclosed in WO 02/051841, WO 03/053966, and WO 2008/012290, which are incorporated herein by reference in their entireties.

Further M3 receptor antagonists having a high potency and long duration of action that, once adsorbed, are degraded to inactive compounds which are deprived of any systemic side effects typical of muscarinic antagonists, are the subject-matter of WO 2010/072338, which is incorporated herein by reference in its entirety.

Such M3 antagonists, belonging to the class of the aminoester derivatives, turned out to be particularly selective and endowed with a high potency. Therefore, these compounds may provide significant therapeutic benefit in the treatment of respiratory diseases such as asthma and COPD, when administered by inhalation.

In order to reach the respiratory tract, among other means, such drugs could be administered through nebulization as liquid formulations. Liquid formulations, in particular aqueous solution formulations, are easy to administer as they are inhaled during normal breathing through a mouth-piece or a face-mask. However, aminoester derivatives may suffer from problems of chemical stability in aqueous solution.

Thus, there remains a need for improved formulations for the administration of antimuscarinic drug by inhalation.

### SUMMARY OF THE INVENTION

Accordingly, it is one object of the present invention to provide novel formulations for the administration of antimuscarinic drug by inhalation.

It is another object of the present invention to provide novel liquid propellant-free formulations for administration through nebulization comprising a M3 antagonist belonging to the class of the aminoester derivatives as active ingredient, wherein said active ingredient is chemically stable upon storage.

Optimally, said formulation shall also exhibit adequate physical stability in the container before use and shall give rise to a good respirable fraction of the active ingredient.

These and other objects, which will become apparent during the following detailed description, have been achieved by the inventors' discovery that liquid propellant-free pharmaceutical formulations for administration through nebulization, which comprise an aminoester derivative of general formula (I), shown below, acting as muscarinic receptor antagonist, in which the compound is dissolved in a solvent comprising at least 75% v/v of water and an optional co-solvent miscible with water; and wherein the pH of the solution is comprised between 3.0 and 5.5,

are effective for the administration of the aminoester derivative.

Thus, in a first embodiment, the present invention provides a liquid propellant-free pharmaceutical formulation for administration through nebulization, comprising an aminoester derivative of general formula (I), shown below, acting as muscarinic receptor antagonist, said compound being dissolved in a solvent comprising at least 75% v/v of water and an optional co-solvent miscible with water; and wherein the pH of the solution is comprised between 3.0 and 5.5.

In a second embodiment, the present invention provides processes for the preparation of the aforementioned formulation.

In a third embodiment, the present invention provides vials which are filled with the present liquid propellant-free formulation.

In a fourth embodiment, the present invention relates to the present liquid propellant-free formulation for use for the prevention and/or treatment of an inflammatory or obstructive airways disease such as asthma or chronic obstructive pulmonary disease (COPD).

In a fifth embodiment, the invention provides methods of preventing and/or treating an inflammatory or obstructive airways disease such as asthma or chronic obstructive pulmonary disease (COPD), which comprises administration by inhalation of an effective amount of a liquid propellant-free formulation according to the present invention.

In a sixth embodiment, the invention relates to the use of the present liquid propellant-free formulation in the manufacture of a medicament for the prevention and/or treatment of an inflammatory or obstructive airways disease such as asthma or chronic obstructive pulmonary disease (COPD).

In a seventh embodiment, the invention provides kits, which comprise:

a) the liquid propellant-free formulation of the invention;

b) a vial containing said formulation; and

c) a nebulizer.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a plot of the solubility of the active ingredient C1 in water-ethanol mixtures as a function of ethanol concentration.

### DETAILED DESCRIPTION OF THE PREFERRED EMBODIMENTS

The terms “active drug,” “active ingredient,” “active,” “active substance,” “active compound,” and “therapeutic agent” are used synonymously.

The terms “muscarinic receptor antagonists,” “antimuscarinic drugs,” and “anticholinergic drugs” are used synonymously.

In the present description, unless otherwise provided, the term “halogen” includes fluorine, chlorine, bromine, and iodine atoms.

The expression “aryl” refers to mono-, or bi-, or tri-cyclic ring systems which have 6 to 20 ring atoms, preferably from 6 to 15 and wherein at least one ring is aromatic. The expression “heteroaryl” refers to mono- or bi-cyclic ring systems with 5 to 20 ring atoms, preferably from 5 to 15, in which at least one ring is aromatic and in which at least one ring atom is a heteroatom or heteroaromatic group (e.g. N, NH, S, or O).

Examples of suitable aryl or heteroaryl monocyclic systems include, for instance, thiophene (thiophenyl), benzene (phenyl), pyrrole (pyrrolyl), pyrazole (pyrazolyl), imidazole (imidazolyl), isoxazole (isoxazolyl), oxazole (oxazolyl), isothiazole (isothiazolyl), thiazole (thiazolyl), pyridine (pyridinyl), imidazolidine (imidazolidinyl), furan (furanyl) radicals, and the like.

Examples of suitable aryl or heteroaryl bicyclic systems include naphthalene (naphthyl), biphenylene (biphenylenyl), purine (purinyl), pteridine (pteridinyl), benzotriazole (benzotriazolyl), quinoline (quinolinyl), isoquinoline (isoquinolinyl), indole (indolyl), isoindole (isoindolyl) radicals and the like.

The term “nebulization” refers to the generation of very fine liquid droplets for inhalation to the lungs by means of suitable devices called nebulizers.

By “single therapeutically effective dose” it is meant the quantity of active ingredient administered at one time by nebulization.

The term “water soluble” refers to a solute which is soluble in water according to the European Pharmacopoeia II Ed 5.2 2005, page 565 (which is incorporated herein by reference in its entirety), e.g. that needs 3 ml of solvent to dissolve 100 mg of solute.

The chemical stability of a formulation refers to the length of time at a given temperature that greater than 90%, preferably equal to or greater 95% of the initial amount of the active ingredient is present in the formulation.

“Chemical stability” refers not only to the stability of chemical degradation but also the stability to racemization of any chiral centers.

The expression “respirable fraction” refers to an index of the percentage of active particles which would reach the deep lungs in a patient.

The respirable fraction, also termed fine particle fraction, is evaluated using a suitable in vitro apparatus such as Multistage Cascade Impactor or Multi Stage Liquid Impinger (MLSI) according to procedures reported in common Pharmacopoeias. It is calculated by the ratio between the delivered dose and the fine particle mass (formerly fine particle dose).

The emitted dose is calculated from the amount of active ingredient collected on a filter (Iso_Gard Filter); the delivered dose is calculated from the cumulative deposition in the apparatus, while the fine particle mass is calculated from the deposition on Stages 3 (S3) to filter (AF) corresponding to particles <5.4 microns (see Marple V, et al., J. Aerosol Med., 2004, 17(4), pp. 335-343, which is incorporated herein by reference in its entirety).

The term “ready-to-use preparation for administration by nebulization” refers to a preparation which is administered directly without further handling and is dispersed in air to form an aerosol by means of a nebulizer.

The term “prevention” means an approach for reducing the risk of onset of a disease.

The term “treatment” means an approach for obtaining beneficial or desired results, including clinical results. Beneficial or desired clinical results can include, but are not limited to, alleviation or amelioration of one or more symptoms or conditions, diminishment of the extent of disease, stabilized (i.e. not worsening) state of the disease, preventing the spread of the disease, delay or slowing of disease progression, amelioration or palliation of the disease state, and remission (whether partial or total), whether detectable or undetectable. The term can also mean prolonging survival as compared to expected survival if not receiving treatment.

By the term “propellant-free” it is meant that the present formulations are substantially free of any propellant, such as low-boiling hydrocarbons (methane, propane, etc.), chlorofluorocarbons (freon 14, freon 12, etc.), and hydrofluoroalkanes (HFA134a, HFA227, etc.). By substantially free, it is meant that the present formulations contain no more than 5% v/v, preferably no more than 1% v/v, more preferably no more than 0.5% v/v, even more preferably no more than 0.1% v/v, still more preferably 0% v/v, of the total amount of such propellants.

The liquid propellant-free pharmaceutical formulation of the present invention for administration through nebulization comprises as an active ingredient, a water soluble physiologically acceptable salt of a compound of general formula (I):

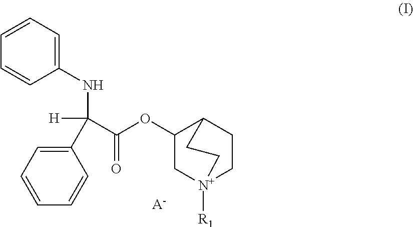

wherein:

R₁ is a group of formula (Y):

—(CH₂)ₚ—P—W (Y)

wherein

p is 0 or an integer of 1 to 4;

P is absent or is selected from the group consisting of O, S, SO, SO₂, and CO;

W is selected from the group consisting of H, aryl, and heteroaryl, wherein aryl and heteroaryl are optionally substituted with one or more substituents selected from the group consisting of halogen atoms, OH, SH, NO₂, CN, COOH, and NH₂; and

A⁻ represents a physiologically acceptable anion.

Advantageously, the physiologically acceptable anion A⁻ is selected from the group consisting of chloride, bromide, iodide, trifluoroacetate, formate, sulfate, phosphate, methanesulfonate, nitrate, maleate, acetate, fumarate, and citrate.

In a first preferred embodiment, p is 1, P is absent, and W is H.

In a second preferred embodiment, p is 1, P is CO, and W is phenyl or thiophenyl.

In a third preferred embodiment, p is 2, P is O, and W is phenyl.

In a fourth preferred embodiment, p is 3, P is O, and W is phenyl.

According to specific embodiments of the invention, specific examples of compounds of formula (I), are shown in Table A.

TABLE A

| Compound | Chemical name and structure |
|---|---|
| C1 | (R)-1-(2-phenoxy-ethyl)-3((R)-2-phenyl-2-phenylamino- |
| | acetoxy)-1-azonia-bicyclo[2.2.2]octane trifluoroacetate |
| | 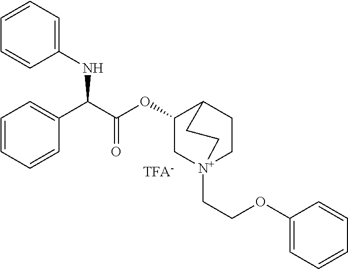 |
| C2 | (R)-1-(2-oxo-2-(thiophen-2-yl)ethyl)-3-(2-phenyl-2- |
| | (phenylamino)acetoxy)-1-azonia-bicyclo[2.2.2]octane |
| | trifluoroacetate |
| | 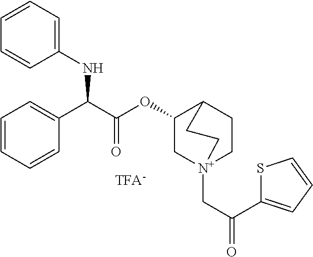 |
| C3 | (R)-1-(3-phenoxypropyl)-3-(2-phenyl-2-(phenylamino) |
| | acetoxy)-1-azonia-bicyclo[2.2.2]octane trifluoroacetate |
| | 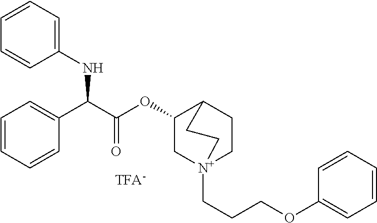 |
| C4 | (R)-1-methyl-3-(2-phenyl-2-(phenylamino)acetoxy)-1-azonia- |
| | bicyclo[2.2.2]octane trifluoroacetate |
| | 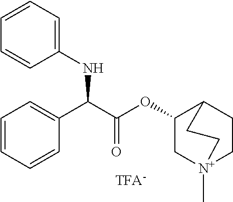 |
| C5 | (R)-1-(2-oxo-2-phenylethyl)-3-(2-phenyl-2-(phenylamino) |
| | acetoxy)-1-azonia-bicyclo[2.2.2]octane chloride |
| | 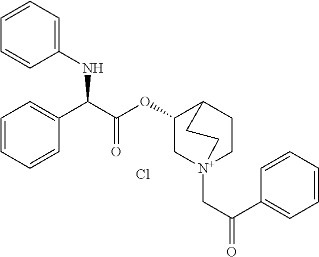 |

The compounds of general formula (I) contain at least two chiral centers that are represented by the carbon atoms marked with asterisks below.

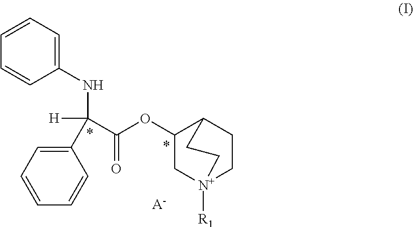

Therefore, formula (I) also encompasses any of the optical stereoisomers, diastereoisomers, and mixtures thereof, in any proportion.

The compounds of general formula (I) may be prepared according to the methods disclosed in WO 2010/072338, which is incorporated herein by reference in its entirety.

The formulations of the present invention exhibit an adequate chemical and physical stability upon storage for pharmaceutical use.

The formulations of the present invention are also able to give rise to a good respirable fraction, typically higher than 50% upon nebulization with common nebulizers.

The solvent wherein the water soluble salt of a compound of general formula (I) should be dissolved can be selected from water or from an aqueous solution comprising at least 75% v/v of water and a co-solvent miscible with water, advantageously 85% v/v water, preferably at least 95% v/v water.

In particular embodiments, the amount of water may be at least 97.5% v/v or at least 98.15% v/v or at least 98.25% v/v.

In another particular embodiment of the invention, the formulation comprises only water as a solvent.

The co-solvent includes, but it is not limited to, polar compounds that contain one or more hydroxyl groups or other polar groups. For example, it includes alcohols, such as ethanol, isopropanol, and glycols including propylene glycol, polyethylene glycol, polypropylene glycol, glycol ether, glycerol, and polyoxyethylene alcohols. In one of the embodiments of the present invention, the preferred co-solvent is ethanol. In other suitable embodiments, the co-solvent may be a mixture of glycerol or propylene glycol or mixtures thereof with ethanol.

The amount of co-solvent shall be adjusted by the person skilled in the art depending on the solubility of the added amount of active ingredient in a particular volume.

In particular embodiments of the present invention, the aqueous solution may comprise 95% v/v of water and 5% v/v ethanol or 97.5% v/v water and 2.5% v/v ethanol, i.e., 95 to 97.5% v/v of water and 5 to 2.5% v/v of ethanol.

If a co-solvent such as glycerol or propylene glycol alone is used, its amount shall be preferably equal to or less than 2.0% v/v, more preferably equal to or lower than 1.85% v/v, even more preferably equal to or lower than 1.75% v/v.

In a particular embodiment, the co-solvent is propylene glycol in an amount of about 1.85% v/v, while in another particular embodiment, the co-solvent is glycerol in an amount of about 1.75% v/v.

The chemical stability of the active ingredient in the formulation has been found herein to be dependent on the pH and the type of buffer.

Therefore, the formulations herein provided should have a pH of 3.0 to 5.5, preferably 3.3 to 5.0, more preferably 3.5 to 4.5.

In certain embodiments, it may be comprised 3.5 to 4.0, while in other embodiments it may be 3.5 or 4.0.

The pH of the formulation shall be adjusted with a buffering agent comprising citric acid including, but not limited to the citric acid/sodium citrate couple (e.g. citrate buffer) and the citric acid/disodium phosphate couple. Citric acid and sodium citrate may be used in the form of hydrates.

The suitable buffer composition in terms of ratio between the acid and the salt, necessary for achieving the claimed pH interval of the invention may be determined empirically using known methods.

As known, the experimental pH value may vary by ±0.1 units.

The buffer concentration for use herein may advantageously vary from 0.1 mM to 20 mM, preferably 2 to 10 mM. In one of the embodiments of the invention, the preferred buffer concentration is 10 mM.

The formulations of the invention may have an osmolality of 240 to 450 mOsm/kg, preferably 250 to 350 mOsm/kg, more preferably 260 to 300 mOsm/kg. Optionally, a tonicity adjusting agent such as sodium chloride or glucose may be added to provide the desired osmolality.

Optionally, the formulation of the invention may comprise further excipients, and additives having no pharmacological activity or at least no undesirable pharmacological activity.

Examples of excipients and additives include, but are not limited to, surfactants, stabilizers, antioxidants, or preservatives which prolong the duration of use of the finished pharmaceutical formulation, flavorings, vitamins, or other known additives. Complexing agents include, but are not limited to, ethylenediaminetetraacetic acid (EDTA) or a salt thereof, such as the disodium salt, citric acid, and salts thereof. Preservatives include, but are not limited to, those that protect the solution from contamination with pathogenic particles, including benzalkonium chloride or benzoic acid, or benzoates such as sodium benzoate. Antioxidants include, but are not limited to, vitamins, ascorbic acid, vitamin E or salts or esters thereof.

The formulation of the present invention may be distributed in suitable containers such as multidose vials or, preferably, unit-dose vials for single dosage administration. Said vials may be made of glass or plastic materials. For example, plastic materials for preparing the unit-dose vials include, but are not limited to, low density polyethylene, high density polyethylene, polypropylene, and polyesters.

The unit-dose vials may be pre-sterilized or, preferably, may be aseptically filled using the “blow, fill and seal” technology. The filling is preferably carried out under an inert atmosphere. Solution formulations can be advantageously sterilized by filtration. Advantageously, the unit-dose vials may have a volume of 1 to 5 ml, i.e., 1 ml, 2 ml, 2.5 ml, 3 ml, 4 ml, or 5 ml, preferably of 1 ml, 2 ml, or 4 ml, more preferably 4 ml.

The formulation of the present invention can also be prepared in a lyophilized form in unitary doses for the reconstitution in a solution. In this alternative embodiment, a single dose of a lyophilized preparation may be reconstituted before use with a solvent in a suitable container as a solution.

The formulation of the invention may be prepared according to known methods.

Typically said methods comprise the steps of:

i) preparing the aqueous solution;

ii) adjusting the pH with a suitable buffer;

iii) adding the active ingredient and mixing to complete dissolution;

iv) optionally adding a further active ingredient;

vi) optionally re-adjusting the pH to the desired value; and

vii) filling the formulation in suitable containers.

The formulations of the present invention are intended for administration by nebulization using suitable apparatus known as nebulizers.

Any nebulizer can be used with the formulation of the invention. Such nebulizers may produce the nebulized droplets by any known method known, including, but not limited to, compressed air (jet), ultrasonic waves, or vibration.

Typically the formulation is administered using a jet nebulizer coupled with a suitable compressor like for example LC PLUS® or LC Sprint® (Pari GmbH, Germany), further optionally coupled with the Akita®Jet inhalation system (Activaero GmbH, Germany) or an ultrasonic nebulizer like for example Aerosonic® (DeVilbiss Healthcare Ltd, UK) and UltraAire® (Omron Ltd, UK), or a mesh-vibrating nebulizer like for example eFlow Rapid® (Pari GmbH, Germany).

The formulation may also be administered by soft-mist inhalers like for example Respimat® (Boehringer Ingelheim GmbH, Germany).

Therefore, the present invention also provides kits, comprising the pharmaceutical formulation provided herein filled in vials for single dosage administration and a nebulizer.

The formulation of the present invention comprises an active ingredient according to general formula (I) in an amount such that, upon dissolution in the solvent, the concentration is 0.001 to 7 mg/ml, advantageously 0.005 to 5 mg/ml, more advantageously 0.0075 to 4 mg/ml, preferably 0.01 to 3 mg/ml, more preferably 0.02 to 2 mg/ml.

In certain embodiments, the concentration may be 0.01 to 4 mg/ml. In further embodiments, it may be 1 to 4 mg/ml. In other embodiments, it may be 0.012 to 3 mg/ml.

For example, when filled in a 4 ml vial and administered by conventional nebulizers, the formulation according to the invention may comprise an amount of 0.004 to 20 mg of an active ingredient according to general formula (I) per 4 ml of the solvent, advantageously 0.02 to 10 mg, preferably 0.04 to 5 mg, more preferably 0.1 to 2.5 mg.

The amount of the active ingredient and hence its single therapeutically active dose will depend on the kind and the severity of the disease and the conditions (weight, sex, age) of the patient.

The amounts are given considering the active ingredient in form of a chloride salt. Depending of the salt used, the amount will vary on the basis of the different molecular weight of the counter ion.

The formulation of the present invention shall be stored at room temperature (25±2° C.) or, preferably, at the refrigerated temperature of 5±3° C., more preferably protected from light.

The formulations of the present invention may further comprise one or more other therapeutic agents useful for the prevention and/or treatment of a respiratory disease, e.g. beta₂-agonists such as salmeterol, milveterol, and vilanterol; corticosteroids such as fluticasone propionate or furoate, flunisolide, mometasone furoate, rofleponide, and ciclesonide; phosphodiesterase-4 (PDE4) inhibitors such as roflumilast; and combinations thereof. Said further active ingredients, depending on their solubility and/or chemical stability in the solvent of the formulation of the present invention, may be present as dissolved substances and/or as suspended particles.

In general terms, the person skilled in the art, on the basis of the available information concerning the stability as well as the water solubility of the active ingredient to be used in combination, shall suitably select those whose chemical stability is compatible with the pH and type of buffer of the formulation of the present invention as well as adjust the percentage of the co-solvent in the aqueous solution in order to achieve their complete dissolution.

Administration of the formulation of the invention may be indicated for prophylactic purposes or for symptomatic relief for a wide range of conditions including respiratory disorders such as chronic obstructive pulmonary disease (COPD) and asthma of all types. Other respiratory disorders for which the formulations of the invention may be beneficial are those characterized by obstruction of the peripheral airways as a result of inflammation and presence of mucus, such as chronic obstructive bronchiolitis, chronic bronchitis, emphysema, acute lung injury (ALI), cystic fibrosis, rhinitis, and adult or respiratory distress syndrome (ARDS).

In addition, the formulation of the invention may be useful in treating smooth muscle disorders such as urinary incontinence and irritable bowel syndrome; skin diseases such as psoriasis; hyperhydrosis and sialorrhea; and gastrointestinal ulcers.

Other features of the invention will become apparent in the course of the following descriptions of exemplary embodiments which are given for illustration of the invention and are not intended to be limiting thereof.

### EXAMPLES

### Example 1

### Determination of the Solubility of the Active Ingredient (R)-1-(2-phenoxy-ethyl)-3-(R)-2-phenyl-2-phenylamino-acetoxy)-1-azonia-bicyclo[2.2.2]octane trifluoroacetate (C1) in Water-Ethanol Mixtures

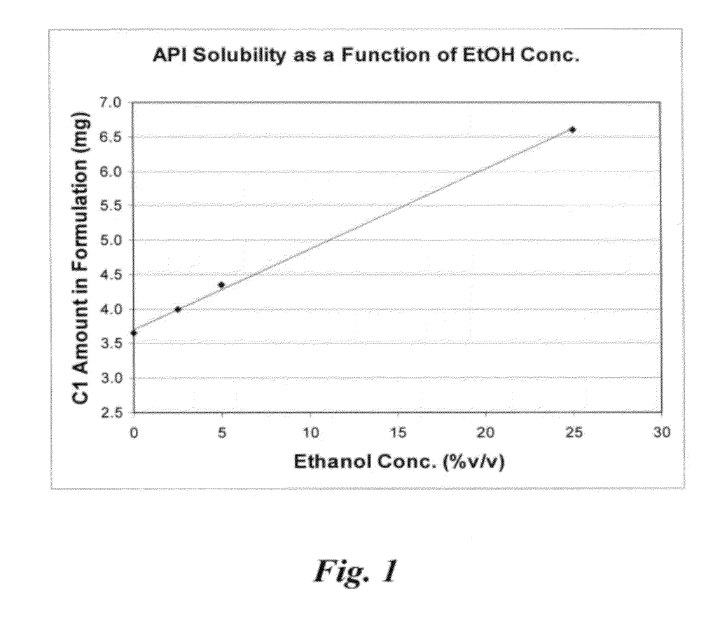

The solubility of C1 in the water/ethanol mixtures was determined as follows. Vials that contained excess of C1 were prepared at 0%, 2.5%, 5%, and 25% ethanol in water. After equilibration, the samples were filtered though a 0.2 μm filter. The results are reported in the plot of FIG. 1, from which the C1 solubility can be extrapolated.

### Example 2

### Propellant-Free Liquid Formulation Comprising (R)-1-(2-phenoxy-ethyl)-3-((R)-2-phenyl-2-phenylamino-acetoxy)-1-azonia-bicyclo[2.2.2]octane trifluoroacetate (C1) as Active Ingredient and Ethanol Solvent

A 10 mM citric acid buffer solution with 5.0% (v/v) ethanol was prepared and the pH was adjusted to 4.5 using 1 N sodium hydroxide. 7 mg of C1 were weighed into a vial, and 2 ml of a pH 4.0 10 mM citric acid buffer solution with 5.0% ethanol was added. The solution was stirred on a vortex 30 seconds every 5 minutes over 45 minutes, then filtered using a 0.2 μm filter. The osmolality turned out be of approximately 290 mOsm/kg.

### Example 3

### Propellant-Free Liquid Formulation Comprising (R)-1-(2-phenoxy-ethyl)-3-((R)-2-phenyl-2-phenylamino-acetoxy)-1-azonia-bicyclo[2.2.2]octane trifluoroacetate (C1) as Active Ingredient and Propylene Glycol as a Co-Solvent

Three 10 mM citric acid buffer solutions with 1.85% (v/v) propylene glycol (PG) were prepared and the pH was adjusted to 4.0, 5.0, and 6.0 using 1 N sodium hydroxide. 2, 3 and 4 mg of C1 were weighed into separate vials, and 1 ml of a pH 4.0 10 mM citric acid buffer solution with 1.85% propylene glycol was added. Each solution was stirred on a vortex 30 seconds every 5 minutes over 45 minutes, then filtered using a 0.2 μm filter. The pH and the osmolality were then tested. Similar formulations comprising C1 as the active ingredient were prepared but using a starting solution wherein the pH was respectively 5.0 and 6.0. All the prepared formulations are reported in Table 1.

TABLE 1
Composition of the formulations.

| | Conc. | Co-solvent | | Citric buffer | Osmolality |
|---|---|---|---|---|---|
| Formulation | (mg/ml) | (% v/v) | pH | (mM) | (mOsm/kg) |
| F1 | 2.0 | 1.85% PG | 4.0 | 10 | 265.0 |
| F2 | 3.0 | 1.85% PG | 4.0 | 10 | 261.0 |
| F3 | 4.0 | 1.85% PG | 4.0 | 10 | 267.0 |
| F4 | 2.0 | 1.85% PG | 5.0 | 10 | 274.0 |
| F5 | 3.0 | 1.85% PG | 5.0 | 10 | 269.0 |
| F6 | 4.0 | 1.85% PG | 5.0 | 10 | 277.0 |
| F7 | 2.0 | 1.85% PG | 6.0 | 10 | 273.0 |
| F8 | 3.0 | 1.85% PG | 6.0 | 10 | 272.0 |
| F9 | 4.0 | 1.85% PG | 6.0 | 10 | 278.0 |

### Example 4

### Stability Studies Carried Out on the Formulations of Example 3

The stabilities of the formulation of Example 3 were evaluated under long-term (25° C., 60% R.H.) conditions. The amount of total degradation products of C1 expressed as percentage by weight, were determined by HPLC. The results are reported in Table 2.

After one week the formulations of the invention at pH 4.0 and 5.0 turned out to be stable, with an amount of degradation products lower than 1.0% for the formulation at pH 4.0 and less than 2.0% for the formulation at pH 5.0. On the contrary, the formulation at pH 6.0 showed already after one week an amount of total degradation products higher than 5%.

TABLE 2
Total degradation products expressed as percentage by weight after
1, 4, and 7 days.

| Formulation | t = 0 | 1 d | 4 days | 7 days |
|---|---|---|---|---|
| F1 | <0.05 | 0.1 | 0.3 | 0.5 |
| F2 | <0.05 | 0.1 | 0.3 | 0.7 |
| F3 | <0.05 | 0.1 | 0.3 | 0.7 |
| F4 | <0.05 | 0.2 | 0.9 | 1.7 |
| F5 | <0.05 | 0.2 | 0.9 | 1.7 |
| F6 | <0.05 | 0.2 | 1.0 | 1.7 |
| F7 | <0.05 | 1.0 | 3.2 | 6.0 |
| F8 | <0.05 | 1.0 | 3.2 | 6.0 |
| F9 | <0.05 | 1.0 | 3.3 | 5.8 |

### Example 5

### Propellant-Free Liquid Formulation Comprising (R)-1-(2-Phenoxy-Ethyl)-3-((R)-2-phenyl-2-phenylamino-acetoxy)-1-azonia-bicyclo[2.2.2]octane trifluoroacetate (C1) as Active Ingredient and Glycerol as a Co-Solvent

Three 10 mm citric acid buffer solutions with 1.75% (v/v) glycerol (Gly) were prepared, and the pH was adjusted to 4.0, 5.0, and 6.0 using 1 N sodium hydroxide. 2, 3, and 4 mg of C1 were weighed into separate vials, and 1 ml of a pH 4.0 10 mM citric acid buffer solution with 1.75% glycerol was added. Each solution was stirred on a vortex 30 seconds every 5 minutes over 45 minutes, then filtered using a 0.2 μm filter. The pH and the osmolality were then tested. Similar formulations were prepared but using a solution wherein the pH was respectively 5.0 and 6.0. All the prepared formulations are reported in Table 3.

TABLE 3
Composition of the formulations.

| | Conc. | Co-solvent | | Citric buffer | Osmolality |
|---|---|---|---|---|---|
| Formulation | (mg/ml) | (% v/v) | pH | (mM) | (mOsm/kg) |
| F10 | 2.0 | 1.75% Gly | 4.0 | 10 | 254.0 |
| F11 | 3.0 | 1.75% Gly | 4.0 | 10 | 262.0 |
| F12 | 4.0 | 1.75% Gly | 4.0 | 10 | 257.0 |
| F13 | 2.0 | 1.75% Gly | 5.0 | 10 | 261.0 |
| F14 | 3.0 | 1.75% Gly | 5.0 | 10 | 270.0 |
| F15 | 4.0 | 1.75% Gly | 5.0 | 10 | 268.0 |
| F16 | 2.0 | 1.75% Gly | 6.0 | 10 | 265.0 |
| F17 | 3.0 | 1.75% Gly | 6.0 | 10 | 270.0 |
| F18 | 4.0 | 1.75% Gly | 6.0 | 10 | 270.0 |

### Example 6

### Stability Studies Carried Out on the Formulations of Example 5

The stabilities of the formulations of Example 5 were evaluated as described in Example 4. The results are reported in Table 4. Also in this case, after one week the formulations of the invention at pH 4.0 and 5.0 turned out to be stable, with an amount of degradation products lower than 1.0% for the formulation at pH 4.0 and less than 2.0% for the formulation at pH 5.0. On the contrary, as reported for the formulation of Example 2, the formulation at pH 6.0 shows already after one week an amount of total degradation products higher than 5%.

TABLE 4
Total degradation products expressed as percentage by weight
after 1, 4, and 7 days.

| Formulation | t = 0 | 1 d | 4 days | 7 days |
|---|---|---|---|---|
| F10 | <0.05 | 0.1 | 0.3 | 0.5 |
| F11 | <0.05 | 0.1 | 0.3 | 0.7 |
| F12 | <0.05 | 0.1 | 0.3 | 0.7 |
| F13 | <0.05 | 0.2 | 1.0 | 1.7 |
| F14 | <0.05 | 0.2 | 0.9 | 1.7 |
| F15 | <0.05 | 0.2 | 0.9 | 1.7 |
| F16 | <0.05 | 1.0 | 3.2 | 8.0 |
| F17 | <0.05 | 1.0 | 3.2 | 7.2 |
| F18 | <0.05 | 1.0 | 3.3 | 7.1 |

### Example 7

### Propellant-Free Liquid Formulation Comprising (R)-1-(2-Phenoxy-Ethyl)-3-((R)-2-phenyl-2-phenylamino-acetoxy)-1-azonia-bicyclo[2.2.2]octane trifluoroacetate (C1) as Active Ingredient and Propylene Glycol as a Co-Solvent at pH 3.5

A 10 mM citric acid buffer solution with 1.85% (v/v) propylene glycol (PG) was prepared, and the pH was adjusted to 3.5. 2.5, 4, 5, and 6 mg of C1 were weighed into separate vials, and 2 ml of a pH 3.5 10 mM citric acid buffer solution with 1.85% propylene glycol was added. Each solution was stirred on a vortex 30 seconds every 5 minutes over 45 minutes, then filtered using a 0.2 μm filter. The pH was then tested. All the prepared formulations are reported in Table 5.

TABLE 5
Composition of the formulations.

| | Conc. | Co-solvent | | Citric buffer |
|---|---|---|---|---|
| Formulation | (mg/ml) | (% v/v) | pH | (mM) |
| F19 | 1.25 | 1.85% PG | 3.5 | 10 |
| F20 | 2.0 | 1.85% PG | 3.5 | 10 |
| F21 | 2.5 | 1.85% PG | 3.5 | 10 |
| F22 | 3.0 | 1.85% PG | 3.5 | 10 |

### Example 8

### Stability Studies Carried Out on the Formulations of Example 7

The stabilities of the formulations of Example 7 were evaluated at 5° C. The amount total degradation products of C1 expressed as percentage by weight, were determined by HPLC. The results are reported in Table 6. After more than 4 days, the formulations of the invention at pH 3.5 turned out to be stable, with an amount of degradation products lower than 0.1.

TABLE 6
Total degradation products expressed as percentage by weight
after 10 hours, 30 hours, and 59 hours.

| Formulation | t = 0 | 10 hours | 30 hours | 59 hours |
|---|---|---|---|---|
| F19 | <0.05 | <0.05 | <0.05 | 0.05 |
| F20 | <0.05 | <0.05 | <0.05 | 0.07 |
| F21 | <0.05 | <0.05 | <0.05 | 0.05 |
| F22 | <0.05 | <0.05 | <0.05 | 0.05 |

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A liquid pharmaceutical formulation, comprising a compound of formula (I): 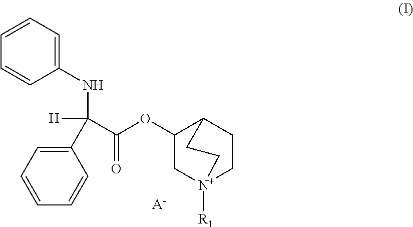
wherein:
R₁ is a group of formula (Y):
—(CH₂)ₚ—P—W (Y)
wherein
p is 0 or an integer of 1 to 4;
P is absent or is selected from the group consisting of O, S, SO, SO₂, and CO;
W is selected from the group consisting of H, aryl, and heteroaryl, wherein aryl and heteroaryl are optionally substituted with one or more substituents selected from the group consisting of a halogen atom, OH, SH, NO₂, CN, COOH, and NH₂;
A⁻ represents a physiologically acceptable anion;
said compound of formula (I) is dissolved in a solvent comprising at least 75% v/v of water and an optional co-solvent, which is miscible with water; and
said formulation has a pH of 3.0 to 5.5.

2. A formulation according to claim 1, wherein said physiologically acceptable anion A⁻ is selected from the group consisting of chloride, bromide, iodide, trifluoroacetate, formate, sulfate, phosphate, methanesulfonate, nitrate, maleate, acetate, citrate, fumarate, tartrate, oxalate, succinate, benzoate, and p-toluenesulfonate.

3. A formulation according to claim 1, wherein said compound of formula (I) is present in a concentration of 0.001 to 7 mg/ml.

4. A formulation according to claim 1, wherein said compound of formula (I) is present in a concentration of 0.005 to 5 mg/ml.

5. A formulation according to claim 1, wherein said compound of formula (I) is present in a concentration of 0.01 to 3 mg/ml.

6. A formulation according to claim 1, which has a pH of 3.5 to 4.5.

7. A formulation according to claim 1, wherein the pH is adjusted by adding a buffering agent which comprises citric acid.

8. A formulation according to claim 1, wherein said optional co-solvent is ethanol.

9. A formulation according to claim 1, wherein said optional co-solvent is glycerol or propylene glycol.

10. A formulation according to claim 1, further comprising another therapeutic agent which is useful for the prevention and/or treatment of a respiratory disease.

11. A formulation according to claim 10, wherein said further therapeutic agent is selected form the group consisting of a beta2-agonist, a corticosteroid, and a phosphodiesterase-4 (PDE4) inhibitor.

12. A method for the prevention and/or treatment of an inflammatory or obstructive airways disease, said method comprising administering an effective amount of a formulation according to claim 1 to a subject in need thereof.

13. A formulation according to claim 12, wherein said disease is asthma or chronic obstructive pulmonary disease (COPD).

14. A vial, which contains a formulation according to claim 1.

15. A kit, comprising:
(a) a vial which contains a formulation according to claim 1; and
(b) a nebulizer.
